(19) United States
(12) Patent Application Publication
(10) Pub. No.: US 2006/0058667 A1
(43) Pub. Date: Mar. 16, 2006

(54) Integrated circuit for an ultrasound system

(76) Inventors: David F. Lemmerhirt, Ann Arbor, MI (US); Collin A. Rich, Ypsilanti, MI (US); Steven M. Martin, Ann Arbor, MI (US); Jeffrey M. Schox, San Francisco, CA (US)

Correspondence Address: SCHOX PLC, 209 N. MAIN STREET #200, ANN ARBOR, MI, 48104, US

(21) Appl. No.: 11/229,197

(22) Filed: Sep. 15, 2005

Related U.S. Application Data
(63) Continuation-in-part of application No. 10/840,548, filed on May 6, 2004.
(60) Provisional application No. 60/610,320, filed on Sep. 15, 2004.
(60) Provisional application No. 60/610,319, filed on Sep. 15, 2004.
(60) Provisional application No. 60/610,337, filed on Sep. 15, 2004.

(51) Int. Cl. A61B 8/14

(52) U.S. Cl. 600/446

(57) ABSTRACT

The preferred embodiment of the invention includes a single integrated circuit for a handheld probe of an ultrasound system. The integrated circuit includes a two-dimensional array of at least 512 transducer cells, each transducer cell is adapted to receive a beam signal, generate an ultrasonic beam, detect an ultrasonic echo at multiple locations, and combine the ultrasonic echoes into a single multiplexed echo signal. Each transducer cell includes at least one ultrasonic beam generator and at least four ultrasonic echo detectors. The integrated circuit also includes a series of beam signal leads adapted to carry the beam signals to the transducer cells and a series of echo signal leads adapted to carry the multiplexed echo signals from the transducer cells.

### CROSS-REFERENCE TO RELATED APPLICATIONS

The present invention claims priority to the following three provisional applications: U.S. Provisional Patent Application No. 60/610,320 filed 15 Sep. 2004 and titled “Beamforming”, U.S. Provisional Patent Application No. 60/610,319 filed 15 Sep. 2004 and titled “Transducer”, and U.S. Provisional Patent Application No. 60/610,337 filed 15 Sep. 2004 and titled “Electronics”.

The present invention also claims priority to U.S. Ser. No. 10/840,548 filed on 06 May 2004 and titled “Ultrasound System Including a Handheld Probe”, which claims priority to the following four provisional applications: U.S. Provisional Patent Application No. 60/468,021 filed 06 May 2003 and titled “Wireless Transducer Head for Medical Ultrasound Systems”, U.S. Provisional Patent Application No. 60/468,022 filed 06 May 2003 and titled “Two-Dimensional Array Design for Three-Dimensional Ultrasonic Imaging”, U.S. Provisional Patent Application No. 60/468,023 filed 06 May 2003 and titled “Medical Ultrasound System Control Integral to the Transducer Head”, and U.S. Provisional Patent Application No. 60/468,024 filed 06 May 2003 and titled “Three-Dimensional Ultrasonic Imaging System.”

Each of the eight applications (the one application and the seven provisional applications) are incorporated in their entirety by this reference.

### TECHNICAL FIELD

This invention relates generally to the medical field, and more specifically to an improved ultrasound system including a handheld probe.

### BACKGROUND

Current medical ultrasound systems typically consist of a transducer head tethered by a communication cable to a central console, which includes the process controls for the ultrasound system. The transducer head generates acoustic waves and detects the reflected echo from the subject being insonified. The central console provides data processing and storage, image display, and/or other such functions typically required during an ultrasound examination.

The communication cable connecting the transducer head and the central console, although a necessity for existing ultrasound systems, can become a significant nuisance to the operator. The cable adds additional weight to the transducer head, which can tire the arm and wrist of the operator after long use. The cable often twists upon itself, which requires the operator—typically a highly paid sonographer or radiologist—to spend time untwisting the cable. Finally, the cable can become entangled with an injured patient or protruding pieces of delicate equipment.

There are, however, several obstacles that prevent the simple substitution of a wireless link for the communication cable of typical medical ultrasound systems. One is related to the physical distance between the transducer head and the central console; the other is related to the hardware of the transducer head.

The length of the communication cable between the transducer head and the central console of typical medical ultrasound systems is relatively short. In this manner, if the operator is holding the transducer head, the operator can reach and operate the process controls (e.g., imaging mode and frame or cine capture) on the central console. If the transducer head were wirelessly linked to the central console, however, the operators would not necessarily be within reach of the central console simply because they are holding the transducer head. Therefore, there exists a need to allow operation of an ultrasound system during a patient examination without requiring physical proximity to the central console.

Since the 1950s, ultrasound imaging has progressed from simple, analog A-mode imaging to far more sophisticated digital B-mode and color Doppler systems. Although these advancements have resulted in high-quality, 2D real-time imagers, an extension of this technology to produce 3D real-time images of comparable or vastly improved quality has not yet been realized. 3D ultrasound would allow medical specialists to view anatomy and pathologic conditions as a volume, thereby enhancing comprehension of the subject patient.

A 2D transducer array is universally acknowledged as the ideal approach for 3D ultrasound image acquisition. A single 3D frame volume from a 2D array, however, may contain gigabytes of raw data from a modest subject (e.g., an 8,000 cm³ frame volume at 0.1 mm resolution) and frame rates for real-time imaging could exceed tens of frames per second. Such large amounts of data cannot be processed in real time by the hardware and software architecture typically used for typical ultrasound systems (e.g., 2D imaging with a 1D array). Collecting, pre-processing, and wirelessly transmitting this amount of data from a handheld probe is not currently possible with a transducer head of typical ultrasound systems.

Thus, there is a need in the medical field to create an improved ultrasound system with a handheld probe that collects enough echo data for 3D imaging and that transmits the echo data by a wireless link.

### BRIEF DESCRIPTION OF THE FIGURES

FIG. 1 is a representation of an ultrasound system of the preferred embodiment;

FIG. 2 is a schematic representation of the central console of the ultrasound system;

FIG. 3 is a schematic representation of a handheld probe for the ultrasound system;

FIG. 4 is a schematic representation of a first example of an integrated circuit for the handheld probe;

FIG. 5 is a representation of the relative size and proportion of the elements of the integrated circuit;

FIGS. 6 and 7 are schematic representations of two variations of a second example of an integrated circuit for the handheld probe; and

FIG. 8 is a representation of an alternative handheld probe for the ultrasound system.

### DESCRIPTION OF THE PREFERRED EMBODIMENT

The following description of the preferred embodiment of the invention is not intended to limit the invention to this preferred embodiment, but rather to enable any person skilled in the art of medical devices to make and use this invention.

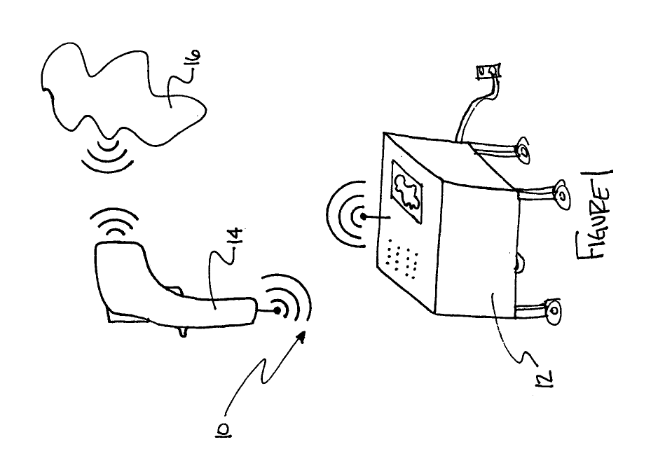

The preferred embodiment of the invention include an ultrasound system, a handheld probe of the ultrasound device, and an integrated circuit for the handheld probe of the ultrasound system. The ultrasound system 10 of the preferred embodiment, as shown in FIG. 1, includes a central console 12 and a handheld probe 14. The handheld probe 14 is adapted to receive a wireless beam signal from the central console 12, generate an ultrasonic beam, detect an ultrasonic echo at multiple locations, combine the ultrasonic echoes into a single multiplexed echo signal, and transmit a multiplexed echo signal to the central console 12. The ultrasound system 10, including the handheld probe and the integrated circuit, provides an improved ultrasound system that collects enough echo data for 3D imaging and that transmits the echo data by a wireless link to overcome the limitations and drawbacks of typical ultrasound systems.

The ultrasound system 10 has been specifically designed to allow medical specialists to view the anatomy and pathologic conditions of a patient. The ultrasound system 10 may, however, be used to view any subject 16 that at least partially reflects ultrasound beams. Such non-medical uses may include ultrasonic microscopy, non-destructive testing, and other situations that would benefit from a volumetric imaging of the subject 16.

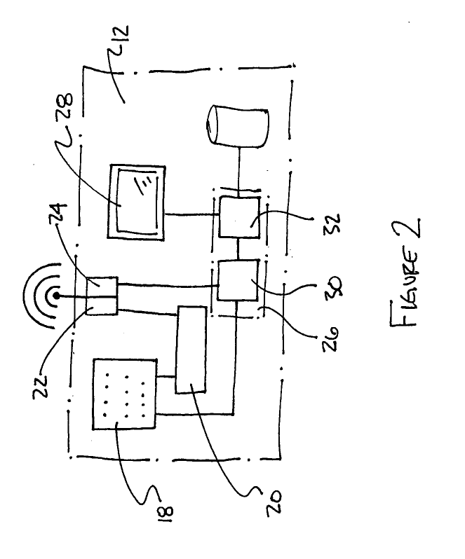

The central console 12 of the preferred embodiment functions to: provide interaction with the operator of the ultrasound system 10; wirelessly communicate with the handheld probe 14; control the ultrasonic beams of the handheld probe 14; process the 3D images from the multiplexed echo signals of the handheld probe 14; and display a 3D image. The central console 12 may further provide other functions, such as providing data storage, data compression, image printouts, format conversions, communication links to a network, or any other appropriate function. To accomplish the five main functions, the central console 12 is conceptually separated into console controls 18, a beam controller 20, a console transmitter 22 and console receiver 24, an image processor 26, and a console display 28, as shown in FIG. 2. The central console 12 is preferably designed as a mobile unit (such as a wheeled cart or a laptop computer), but may alternatively be designed as a fixed unit (such as a cabinet structure).

The console controls 18 of the central console 12 provide interaction with the operator of the ultrasound system 10. The console controls 18 preferably allow the operator to configure the ultrasound system 10, to switch between imaging modes, and to capture frame/cine. The console controls 18 may alternatively provide other appropriate functions. Input from the operator is collected, parsed, and sent to the image processor 26 and/or the beam controller 20 as appropriate. The console controls 18 may include knobs, dials, switches, buttons, touch pads, fingertip sensors, sliders, joysticks, keys, or any other appropriate device to provide interaction with the operator.

The beam controller 20 of the central console 12 controls the ultrasonic beams of the handheld probe 14. The operator of the ultrasound system 10, through the console controls 18 described above, may select a particular imaging mode (e.g., 3D, 2D slice, or local image zoom) for a subject 16. To comply with this selection, the beam controller 20 preferably creates a beam signal that adjusts or modulates the frequency, sampling rate, filtering, phasing scheme, amplifier gains, transducer bias voltages, and/or multiplexer switching of the handheld probe 14. Alternatively, the beam controller 20 may create two or more signals that adjust or modulate these parameters. Further, the beam controller 20 may create a beam signal that adjusts or modulates other appropriate parameters of the handheld probe 14.

The console transmitter 22 and the console receiver 24 of the central console 12 function to provide a wireless communication link with the handheld probe 14. Specifically, the console transmitter 22 functions to transmit beam signals to the handheld probe 14, while the console receiver 24 functions to receive echo signals from the handheld probe 14. In the preferred embodiment, the console transmitter 22 and the console receiver 24 use radiofrequency (RF) communication and an appropriate protocol with a high data throughput. In an alternative embodiment, however, the console transmitter 22 and the console receiver 24 may use infrared or other high-speed optical communication instead of, or in addition to, RF communication. The console transmitter 22 and the console receiver 24 may incorporate frequency hopping, spread-spectrum, dual-band, encryption, and/or other specialized transmission techniques known in the art to ensure data security and/or integrity in noisy environments. In the preferred embodiment, the console transmitter 22 and the console receiver 24 are located within different housings and are operated at different frequencies. In an alternative embodiment, the console transmitter 22 and the console receiver 24 may be combined (as a console transceiver) and/or may operate within the same channel or frequency.

The image processor 26 of the central console 12, which functions to construct 3D images from the multiplexed echo signals of the handheld probe 14, is preferably composed of a frame compiler 30 and an image engine 32. The frame compiler 30 of the image processor 26 functions to assemble a single 3D image (or 3D frame) from the multiplexed echo signals of the handheld probe 14. The echo signals, which are a series of pulses with specific time, amplitude, and phasing information, are correlated, summed, and transformed into voxels for the 3D image. Noise reduction, phase deaberration, contrast enhancement, orthogonal compounding, and other operations are also performed at this stage. In the preferred embodiment, as much as possible, these operations are performed in parallel fashion with dedicated algorithms, thus allowing the frame compiler 30 to be optimized for maximum speed. The frame compiler 30 preferably consists of a massively parallel set of lower-cost, medium-performance DSP cores, but may alternatively include other appropriate devices.

The image engine 32 of the image processor 26 receives complete frames from the frame compiler 30 and provides all higher-level processing (such as image segmentation) of the 3D frames. In the preferred embodiment, the image engine 32 also serves as a collection point for all echo data in the ultrasound system 10. The image engine 32 preferably consists of a high-performance, highly programmable DSP core, but may alternatively include other appropriate devices. In an alternative embodiment, the image processor 26 may include other appropriate devices to construct 3D images from the multiplexed echo signals of the handheld probe 14.

The console display 28 functions to present an image of the subject 16 to the operator in a form that facilitates easy and intuitive manipulation, navigation, measurement, and quantification. Examples of display modes include 3D, semi-transparent rendering, and 2D slices through the 3D structure. The console display 28 preferably includes a conventional LCD screen, but may alternatively include any appropriate device (such as a holographic or stereoscopic device) to present the scanned images.

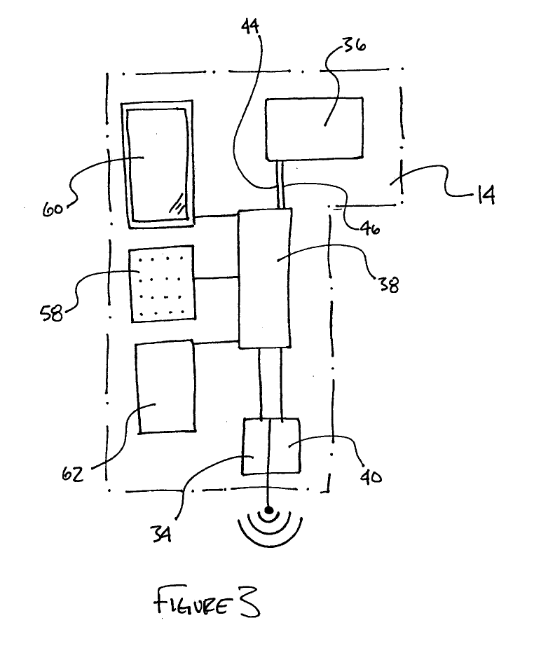

The handheld probe 14 of the preferred embodiment functions to: wirelessly receive beam signals from the central console 12; generate an ultrasonic beam and detect an ultrasonic echo at multiple locations; combine the ultrasonic echoes into a single multiplexed echo signal; and wirelessly transmit the echo signals to the central console 12. The handheld probe 14 may further provide other functions, such as providing data storage, data compression, or any other appropriate function. To accomplish the four main functions, the central console 12 is conceptually separated into a probe receiver 34, a first integrated circuit 36, a second integrated circuit 38, and a probe transmitter 40, as shown in FIG. 3.

The probe receiver 34 and the probe transmitter 40 of the handheld probe 14 function to provide a wireless communication link with the central console 12. Specifically, the probe receiver 34 functions to receive beam signals from the central console 12, while the probe transmitter 40 functions to transmit a multiplexed echo signal to the central console 12. The probe receiver 34 and the probe transmitter 40 use the same communication method and protocol as the console transmitter 22 and the console receiver 24. In the preferred embodiment, the probe receiver 34 and the probe transmitter 40 are located within different housings. In an alternative embodiment, the probe receiver 34 and the probe transmitter 40 may be combined (as a probe transceiver).

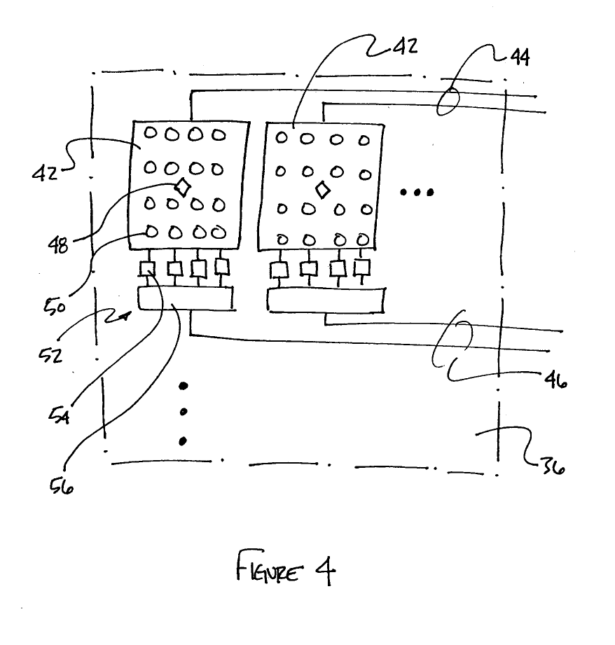

The first integrated circuit 36 of the handheld probe 14 functions to generate an ultrasonic beam, detect an ultrasonic echo at multiple locations, and to combine the ultrasonic echoes into multiplexed echo signals. The first integrated circuit 36 preferably accomplishes these functions with the use of a 2D array of transducer cells 42, a series of beam signal leads 44 that are adapted to carry the beam signals to the transducer cells 42, and a series of echo signal leads 46 that are adapted to carry the multiplexed echo signals from the transducer cells 42, as shown in FIG. 4. The first integrated circuit 36 may alternatively accomplish these functions with other suitable devices.

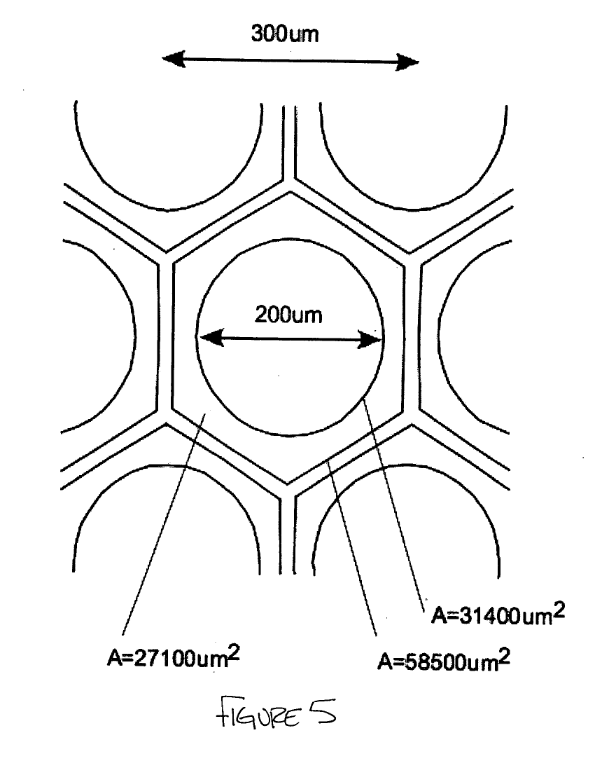

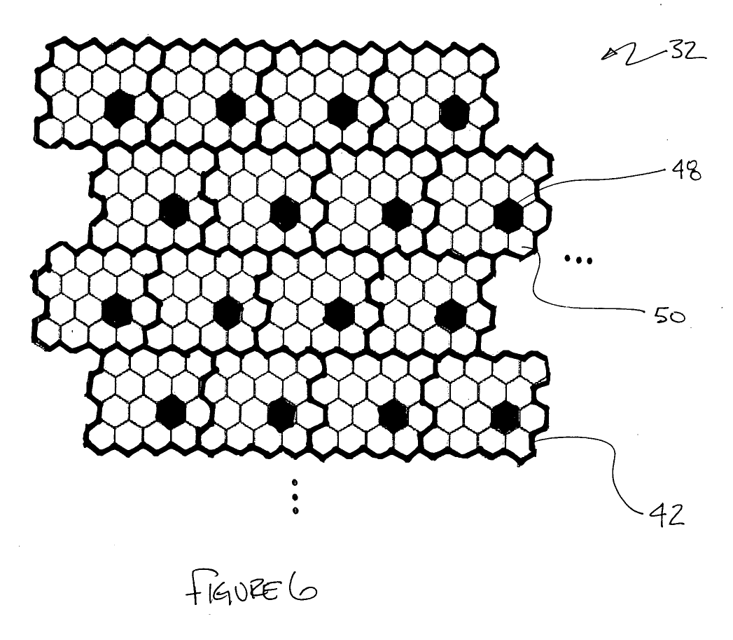

Each transducer cell 42 of the first integrated circuit 36, which functions as a 2D phased subarray to scan one sector of the entire viewing field, preferably includes at least one ultrasonic beam generator 48, at least four (and preferably fifteen or sixteen) ultrasonic echo detectors 50, and at least one first multiplexer 52. The ultrasonic beam generator 48 and the ultrasonic echo detectors 50 of the transducer cell 42 function to generate an ultrasonic beam and to detect an ultrasonic echo at multiple locations, respectively. Preferably, the ultrasonic beam generator 48 and the ultrasonic echo detectors 50 are separate elements, which simplifies the front-end electronics for the first integrated circuit 36 and allows the ultrasonic beam generator 48 and the ultrasonic echo detectors 50 to be separately optimized for their individual function. For example, the ultrasonic beam generator 48 may be optimized for high output (with increased ruggedness), while the ultrasonic echo detector 50 may be optimized for high sensitivity. This separate optimization may reduce edge wave effects (since a single point source can be fired instead of a complete subaperture). Although separate elements, the ultrasonic beam generator 48 and the ultrasonic echo detector 50 preferably share a basic shape and construction and preferably differ only by the diaphragm diameter, thickness, tensile stress, gap spacing, control electronics, and/or electrode configuration. Alternatively, the ultrasonic beam generator 48 and the ultrasonic echo detectors 50 may be formed as the same component (i.e., dual-function transducers). If the first integrated circuit 36 is operating at 3 MHz, the ultrasonic beam generator 48 and the ultrasonic echo detectors 50 have a preferred diameter of 100-200 μm and a preferred pitch of approximately 250±50 μm, as shown in FIG. 5. The ultrasonic beam generator 48 and the ultrasonic echo detectors 50 may, however, have any suitable diameter and pitch.

The first multiplexer 52 of the transducer cell 42 functions to combine the ultrasonic echoes from the ultrasonic echo detectors 50 into a multiplexed echo signal. To collect enough echo data for 3D imaging, the first integrated circuit 36 preferably includes at least 4,096 ultrasonic echo detectors 50, more preferably includes at least 15,360 ultrasonic echo detectors 50, and most preferably includes at least 16,384 ultrasonic echo detectors 50. From a manufacturing standpoint, the number of echo signal leads 46 between the first integrated circuit 36 and the second integrated circuit 38 is preferably equal to or less than 1024 connections, and more preferably equal to or less than 512 connections. Thus, the first multiplexer 52 preferably combines the echo signals at least in a 4:1 ratio. The first multiplexer 52 may use time division multiplexing (TDM), quadrature multiplexing, frequency division multiplexing (FDM), or any other suitable multiplexing scheme. Further, the first multiplexer 52 may actually be two multiplexers (indicated in FIG. 4 as a first portion 54 and a second portion 56) combined that either use the same or different multiplexing schemes.

In a first example of the preferred embodiment, the transducer cell 42 is square shaped and the first integrated circuit 36 includes 1,024 transducer cells 42 (preferably arranged in a square pattern with thirty-two transducer cells 42 along one dimension and thirty-two transducer cells 42 along another dimension). Preferably, each transducer cell 42 includes: sixteen ultrasound echo detectors 50 (plus one ultrasound beam generator 48 and one first multiplexer 52) in a transducer cell, and 1,024 transducer cells 42 in the first integrated circuit 36. This arrangement provides a manageable level of echo signal leads 46 to the second integrated circuit 38 (1,024 echo signal leads), while providing enough echo data (16,384 ultrasonic echo detectors 50) for 3D image rendering. The first multiplexer 52, in this arrangement, combines sixteen echo signals into one multiplexed echo signal using a 16:1 TDM device. In a variation of this example, the first multiplexer 52 combines only four echo signals into one multiplexed echo signal using a 4:1 TDM device. Since there are four multiplexed echo signals and only one echo signal lead, the first integrated circuit of this example performs four passes, each pass with a new beam signal and each pass with only ¼^{th} of the ultrasonic echo detectors 50 contributing to the echo signal. In this manner, the first multiplexer 52 is only combining a portion of the echo signals into a multiplexed signal.

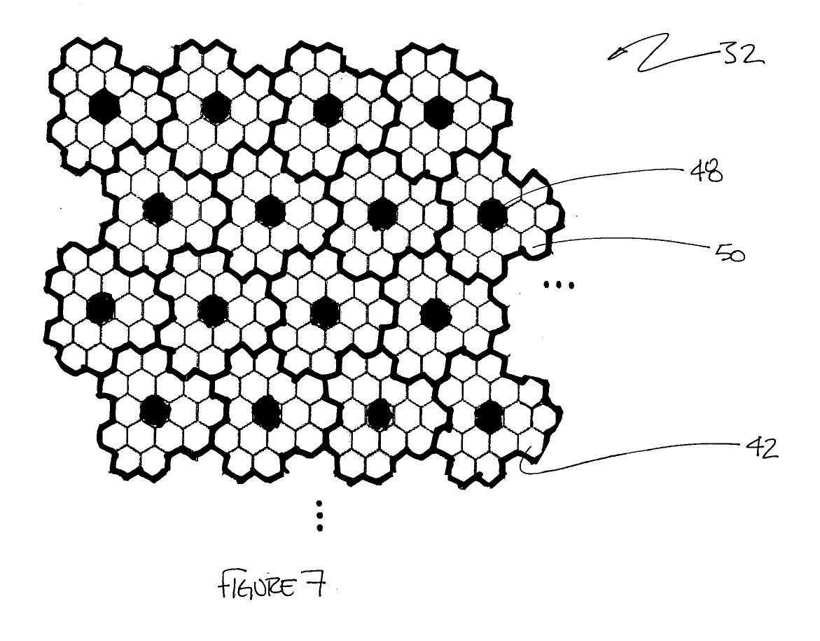

In a second example of the preferred embodiment, as shown in FIG. 5, the transducer cell 42 is roughly rectangular shaped and the first integrated circuit 36 includes 1,024 transducer cells 42 (preferably arranged in a square pattern with thirty-two transducer cells 42 along one dimension and thirty-two transducer cells 42 along another dimension). Preferably, each roughly rectangular transducer cell 42 includes: one ultrasound beam generator 48 near the center, fifteen ultrasound echo detectors 50, and one first multiplexer (not shown). The ultrasound beam generators 48 are preferably arranged in a regular hexagonal tessellation, but may alternatively be arranged in any suitable pattern. This arrangement provides a manageable level of echo signal leads to the second integrated circuit (1,024 echo signal leads), while providing enough echo data (15,360 ultrasonic echo detectors 50) for 3D image rendering. The first multiplexer, in this arrangement, combines fifteen echo signals into one multiplexed echo signal using a 15:1 TDM device (potentially implemented as a 16:1 device, or as two 4:1 devices, with one repeated or null signal). In a variation of this second example, as shown in FIG. 7, the transducer cell 42 is roughly snowflake shaped. Preferably, each roughly snow-flaked shaped transducer cell 42 includes: one ultrasound beam generator 48 in the center, fifteen ultrasound echo detectors 50 (arranged as six “interior” ultrasound echo detectors 50 and nine “exterior” ultrasound echo detectors 50), and one first multiplexer (not shown).

Since the first integrated circuit 36 is preferably limited to electronics that are essential to getting signals on- and off-chip, the first integrated circuit 36 may be manufactured by a standard low-cost CMOS process at an existing foundry (e.g. AMI Semiconductor, 1.5 μm). The ultrasonic beam generator 48 and the ultrasonic echo detectors 50 are preferably microfabricated on the first integrated circuit 36 as capacitive micro-machined ultrasonic transducers (cMUT), similar in structure and function to devices disclosed by U.S. Pat. No. 6,246,158 (which is incorporated in its entirety by this reference), but differing significantly in structural materials and manufacturing method.

The mechanical structure of the cMUT device is preferably formed by layers deposited and patterned as part the foundry CMOS process itself (and preferably not augmented with additional steps for depositing material, aligning and patterning layers, and releasing/sealing diaphragms). The steps performed on the first integrated circuit 36 after the foundry fabrication preferably include only blanket etch and deposition steps, which require no alignment procedure or only rough alignment (with tolerances greater than 400 μm).

Preferably, the structure consists of a polysilicon lower electrode along with a dielectric membrane and metal upper electrode, with all layers integral to the CMOS process flow. In this case, a capacitive gap with a thickness of 0.5 μm to 1.2 μm is preferably formed between the membrane and the lower electrode by selectively etching a sacrificial metal layer (also integral to the CMOS process) that has been patterned to be exposed when the chip is immersed in a metal etch solution after completion of the foundry CMOS process. A vacuum-sealed cavity is preferably formed between the membrane and the lower electrode by sealing access ports to the gap using a low-temperature blanket dielectric deposition under vacuum (by PECVD and/or sputtering).

Alternatively, the structure may include a metal lower electrode and a dielectric membrane formed within the CMOS process flow. A gap is preferably formed between the dielectric membrane and the lower electrode by selectively etching a sacrificial metal layer (also integral to the CMOS process) that has been patterned to be exposed to attack when the chip is immersed in a metal etch solution after completion of the foundry CMOS process. In this case, vacuum sealing and the formation of the upper electrode, which is electrically common to all membranes on the chip, are accomplished by blanket depositions of metal and dielectric layers under vacuum (by PECVD and/or sputtering).

The second integrated circuit 38, as shown in FIG. 3, of the handheld probe 14 functions to receive and transmit the beam signals from the probe receiver 34 to the beam signal leads 44 of the first integrated circuit 36, and to receive and transmit the multiplexed echo signals from the echo signal leads 46 to the probe transmitter 40. Preferably, the second integrated circuit 38 further conditions the multiplexed echo signals to facilitate wireless communication to the central console 12. The conditioning may include converting the analog echo signals to adequately sampled (e.g. above Nyquist) digital signals, amplifying the analog echo signals, compressing the digital echo signals, and performing an error-correction process on the echo signals. The conditioning may further include additional multiplexing of the multiplexed echo signals into one channel (or simply less channels). Any number of multiplexing schemes may be used, including time-division multiplexing, code-division multiplexing, frequency-division multiplexing, packet-based transmission, or any other suitable multiplexing scheme. The second integrated circuit 38 preferably uses conventional devices and manufacturing methods, but may alternatively use any suitable device and any suitable manufacturing method.

In the preferred embodiment, the handheld probe 14 further provides time gain compensation of the echo signals, which corrects for attenuation and allows objects at a greater depth to be clearly depicted with objects of lesser depth. This function may be integrated onto the first integrated circuit 36, the second integrated circuit 38, or any other suitable locations within the handheld probe 14. In alternative embodiments, the problem of attenuation may be solved with other suitable devices, either within the handheld probe 14, the central console 12, or any other suitable location.

In the preferred embodiment, the central console 12 transmits multiple beam signals as a single multiplexed beam signal. For this reason, the central console 12 preferably includes a multiplexer (not shown) and the handheld probe 14 includes a de-multiplexer (not shown). In alternative embodiments, the beam signals are sent using multiple channels or using another suitable scheme.

In the preferred embodiment, the handheld probe 14 further includes probe controls 58, which function to provide additional interaction with the operator of the ultrasound system 10. Like the console controls 18, the probe controls 58 preferably allow the operator to configure the ultrasound system 10, to switch between imaging modes, and to capture frame/cine. Because of the proximity to the subject 16, however, the probe controls 58 may further include additional features, such as flag image, add caption or notation, add voice notation, and take measurement from image. The probe controls 58 may alternatively provide other appropriate functions. Input from the operator is collected, wirelessly transmitted to the central console 12, and routed to the image processor 26 and/or the beam controller 20 as appropriate. The probe controls 58 may include knobs, dials, switches, buttons, touch pads, fingertip sensors, sliders, joysticks, keys, or any other appropriate device(s) to provide interaction with the operator. The handheld probe 14 with the probe controls 58 of the preferred embodiment satisfies the need to allow operation of an ultrasound system 10 during a patient examination without requiring physical proximity to the central console 12.

In the preferred embodiment, the handheld probe 14 further includes a probe display 60. In a first variation of the preferred embodiment, the console transmitter 22 and the probe receiver 34 are further adapted to communicate information about the system configuration (such as imaging modes). With this variation, the probe display 60 is preferably adapted to display the system configuration. In a second variation of the preferred embodiment, the console transmitter 22 and the probe receiver 34 are further adapted to communicate a processed image of the subject 16 (e.g., 3D, semi-transparent rendering, and 2D slices through the 3D structure). With this variation, the probe display 60 is preferably adapted to display the processed image. In a third variation, the console transmitter 22 and the probe receiver 34 are adapted to communicate both the information about the system configuration and the processed images. With this variation, the handheld probe 14 may include an additional probe display 60, or may include a switch between the two sources. The probe display 60 preferably includes a conventional LCD screen, but may alternatively include any appropriate device such as individual lights, digital displays, alphanumeric displays, or other suitable indicators. With the probe controls 58 and the probe display 60, the handheld probe 14 of the preferred embodiment further exceeds the need to allow operation of an ultrasound system 10 during a patient examination without requiring physical proximity to the central console 12.

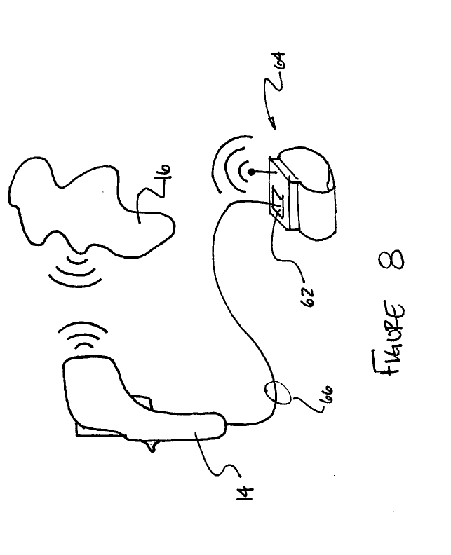

In the preferred embodiment, the handheld probe 14 further includes a power source 62, which functions to power the components of the handheld probe 14. The power source 62 is preferably a conventional rechargeable battery, but may alternatively be a capacitor, a fuel cell, or any other suitable power source 62. Considering the state of battery technology, however, it is possible that the addition of a power source 62 would make the handheld probe 14 unacceptably heavy or bulky. Thus, in a variation of the preferred embodiment shown in FIG. 8, the power source 62 is located in a remote portion 64 of the handheld probe 14, which is connected to the handheld probe 14 with a lightweight cord 66. The remote portion 64 may be designed to be strapped to the operator's body (e.g., wrist, arm, or shoulder) or clipped to the operator's belt, with the cable routed such that it is kept conveniently out of the way (e.g., along the arm). Although this variation still requires a cable connected to the handheld probe 14, the cable moves with the operator and thus provides a degree of freedom that is still greater than a transducer head tethered to the central console. Further, in the variation of the preferred embodiment, other elements of the handheld probe 14 may be located in the remote portion 64. For example, the probe receiver, the probe transmitter, the probe controls, and/or the probe display may be located in the remote portion 64 of the handheld probe 14.

As a person skilled in the art of ultrasound systems will recognize from the previous detailed description and from the figures and claims, modifications and changes can be made to the preferred embodiment of the invention without departing from the scope of this invention defined in the following claims.

We claim:

1. A single integrated circuit for a handheld probe of an ultrasound system, comprising:
a two-dimensional array of at least 512 transducer cells, each transducer cell adapted to receive a beam signal, generate an ultrasonic beam, detect an ultrasonic echo at multiple locations, and combine the ultrasonic echoes into a single multiplexed echo signal, wherein each transducer cell includes at least one ultrasonic beam generator and at least four ultrasonic echo detectors;
a series of beam signal leads adapted to carry the beam signals to the transducer cells; and
a series of echo signal leads adapted to carry the multiplexed echo signals from the transducer cells.

2. The integrated circuit of claim 1, wherein the two-dimensional array includes at least 1024 transducer cells, and wherein each transducer cell includes at least fifteen ultrasonic echo detectors.

3. The integrated circuit of claim 1, wherein the ultrasonic beam generator is a separate element from the at least four ultrasonic echo detectors.

4. The integrated circuit of claim 3, wherein the ultrasonic beam generator and the at least four ultrasonic echo detectors are each an ultrasonic transducer capable of both generating an ultrasonic beam and detecting an ultrasonic echo.

5. The integrated circuit of claim 4, wherein the ultrasonic beam generator is capable of generating an ultrasonic beam with a higher output than one of the ultrasonic echo detectors.

6. The integrated circuit of claim 5, wherein one of the ultrasonic echo detectors is capable of detecting an ultrasonic echo with greater sensitivity than the ultrasonic beam generator.

7. The integrated circuit of claim 3, wherein the ultrasonic beam generator and the at least four ultrasonic echo detectors differ by at least one characteristic from a group consisting of diaphragm diameter, thickness, tensile stress, gap spacing, control electronics, and electrode configuration.

8. The integrated circuit of claim 1, wherein the ultrasound beam generators are arranged in a substantially regular hexagonal tessellation.

9. The integrated circuit of claim 7, wherein each ultrasound beam generator is arranged in the center of the transducer cell.

10. The integrated circuit of claim 1, wherein each transducer cell includes a multiplexer adapted to combine at least a portion of the ultrasonic echoes into a single multiplexed echo signal.

11. A handheld probe for an ultrasound system, comprising:
a probe receiver adapted to wirelessly receive beam signals;
the first integrated circuit of claim 1;
a second integrated circuit including a multiplexer connected to the series of echo signal leads and adapted to combine the multiplexed echo signals; and
a probe transmitter connected to the multiplexer and adapted to wirelessly transmit a multiplexed echo signal.

12. The handheld probe of claim 11 further comprising a display.

13. The handheld probe of claim 11, wherein the two-dimensional array includes at least 1024 transducer cells, and wherein each transducer cell includes at least fifteen ultrasonic echo detectors.

14. An ultrasound system, comprising:
a central console including a controller adapted to create beam signals; a console transmitter connected to the controller and adapted to wirelessly transmit the beam signals; and
the handheld probe of claim 11;
wherein the central console further includes a console receiver adapted to wirelessly receive a multiplexed echo signal; and an image processor adapted to construct an image from the echo signals.

15. The ultrasound system of claim 14 further comprising a display.

16. The ultrasound system of claim 14, wherein the two-dimensional array includes at least 1024 transducer cells, and wherein each transducer cell includes at least fifteen ultrasonic echo detectors.
